(19) United States
(12) Patent Application Publication
(10) Pub. No.: US 2023/0405280 A1
(43) Pub. Date: Dec. 21, 2023

(54) NEEDLE ASSEMBLIES AND RELATED METHODS

(71) Applicant: B. BRAUN MELSUNGEN AG, Melsungen (DE)

(72) Inventors: Lilian Zhi Ling Lew, Georgetown, Penang (MY); Teng Lip Khoo, Kepala Batas, Penang (MY)

(73) Assignee: B. BRAUN MELSUNGEN AG, Melsungen (DE)

(21) Appl. No.: 18/247,023

(22) PCT Filed: Oct. 5, 2021
(86) PCT No.: PCT/EP2021/077417

§ 371 Date: Mar. 28, 2023

Related U.S. Application Data
(60) Provisional application No. 63/088,139, filed on Oct. 6, 2020.

(51) Int. Cl. A61M 25/06; A61M 25/00

(52) U.S. Cl. CPC A61M 25/0606; A61M 25/0097; A61M 25/0009

(57) ABSTRACT

The present disclosure relates to catheter devices in which a bushing is disposed in an interior cavity of a catheter hub. A catheter tube and a reinforcement sleeve are sleeved over the bushing and the combination catheter tube and reinforcement sleeve wedged to the catheter hub. The reinforcement sleeve overlaps with the catheter tube at least at a distal end of the catheter hub, and at a proximal end of the catheter tube. A needle hub with a needle projects through the bushing and the catheter tube in a ready to use position.

### FIELD OF ART

The invention relates generally to needle devices, systems, and methods for use where medicines are delivered vascularly. More particularly, the present disclosure relates to catheter devices or assemblies and needle configurations used in intravenous medical devices and methods for using and making such devices and systems.

### BACKGROUND

Generally, vascular access devices are used for communicating fluids with the vascular system of patients. For example, catheters are used for infusing fluid, such as normal saline solution, various medicaments, and total parenteral nutrition, into a patient, withdrawing blood from a patient, or monitoring various parameters of the patient's vascular system.

A common type of intravenous (IV) catheter is an over-the-needle peripheral IV catheter. As its name implies, an over-the-needle catheter tube is mounted over an introducer needle having a sharp distal tip. At least the inner surface of the distal portion of the catheter tube tightly wraps or surrounds the outer surface of the needle to prevent peel-back of the catheter tube during insertion of the catheter into the blood vessel. The catheter tube and the introducer needle are assembled so that the needle tip of the introducer needle extends beyond the distal tip of the catheter tube with the bevel of the needle facing up away from the patient's skin. The catheter material can be partially transparent and can have stripes of transparent material and opaque stripes for providing x-ray contrast. The catheter and introducer needle are generally inserted at a shallow angle through the patient's skin into a blood vessel.

In order to verify proper placement of the needle and catheter in the blood vessel, the clinician generally looks for blood flashback as confirmation of the access. The first blood flashback is through the needle and into a transparent needle hub, which is sometimes referred to as primary blood flashback. This confirms at least the needle has found the vein. Then as the needle is withdrawn in a proximal direction away from the catheter tube, the blood will flash back between the needle and the catheter tube. This is sometimes referred to as secondary flashback, which confirms that the catheter tube has found the vein. Once proper placement of the catheter into the blood vessel is confirmed, the clinician may apply pressure to the blood vessel by pressing down on the patient's skin over the blood vessel distal of the introducer needle and the catheter. This finger pressure occludes the vessel, minimizing further blood flow through the catheter and possibly leaking out the catheter hub.

In some IV catheter assemblies, the needle has an open notch, through which blood can flow into the space between the needle and catheter tube. This “instant flash” confirms only that the needle tip has entered the vein but not necessarily that the catheter tube has entered the vein. Because there is first blood between the needle and the catheter tube when a notch is employed, a secondary flashback is not possible.

The clinician may then withdraw the introducer needle from the catheter. The introducer needle may be withdrawn into a needle tip shield or needle cap that covers the needle tip and prevents accidental needle sticks. When the needle has an open notch, the blood between the distal opening and the open notch is not held by capillary action and can drip from the needle.

After the needle is withdrawn from the catheter, the catheter hub may then be taped against the skin of the patient to firmly secure the catheter hub in place with the catheter tube accessing the blood vessel through an insertion point. Often times, the axis of the catheter tube may not be aligned with and/or is off centered from the centerline of the catheter hub after the catheter hub has been secured to the patient. The difference in alignment between the catheter tube and the centerline of the catheter hub can cause the flexible tube to form a tight bend, which can affect the patient's comfort at the insertion point, restrict flow through the catheter tube at the bend, or form a kink along the catheter tube. Any movement from the patient such as, for example, an extension or flexion of the muscle near the insertion point, can increase discomfort to the patient and can cause a kink to form in the catheter tube.

### SUMMARY

The various embodiments of a needle assembly have several features, no single one of which is solely responsible for their desirable attributes. Without limiting the scope of the present embodiments as set forth in the claims that follow, their more prominent features now will be discussed briefly.

A catheter assembly provided in accordance with aspects of the present disclosure may include a catheter hub having a hub body defining an interior cavity. The catheter assembly may further include a bushing disposed in the interior cavity. The catheter assembly may further include a catheter tube sleeved over the bushing. The catheter assembly may further include a sleeve sleeved over the bushing. The sleeve may overlap with the catheter tube at least at a distal end of the catheter hub. The catheter assembly may further include a needle hub with a needle including a needle shaft having a needle lumen and a needle tip. The needle shaft may project through the bushing and the catheter tube in a ready to use position.

The catheter tube and the sleeve may have a first and second length, respectively, along a needle shaft projectile direction. The first length may be greater than the second length. The catheter tube or the sleeve may be in direct contact with the bushing.

The sleeve may be disposed over or within the catheter tube. The sleeve may have a larger diameter when the sleeve is over the catheter tube than when the sleeve is within the catheter tube.

The catheter tube or the sleeve may be a heat shrink tube.

The bushing may further include a tapered seat coupled to the catheter tube or the sleeve. The tapered seat may be conical with a wider opening at a proximal end and a relatively narrower opening at a distal end. The bushing may be metal.

The catheter assembly may further include a needle guard having a proximal wall with a proximal opening and two resilient arms slidably on the needle shaft.

The catheter assembly may further include a valve for limiting fluid flow through the catheter hub.

Another aspect of the present disclosure is a method for manufacturing a catheter assembly. The method may include forming a catheter hub with an interior cavity. The method may further include sleeving a catheter tube over a bushing. The method may further include sleeving a sleeve over the bushing. The method may further include securing the bushing in the interior cavity of the catheter hub. The method may further include forming a needle hub with a needle. The needle may include a needle shaft having a needle lumen and a needle tip. The needle shaft may project through the bushing and the catheter tube.

The method may further include placing a needle guard having a proximal wall with a proximal opening and two resilient arms slidably on the needle shaft.

The method may further include placing a valve for limiting fluid flow through a catheter hub within the interior cavity of the catheter hub.

A still further aspect of the invention is a method for resisting catheter tube kinking comprising sleeving a non-metallic reinforcement sleeve onto a distal section of a bushing in addition to sleeving a catheter tube onto the distal section and securing the bushing to an interior of a catheter hub. The catheter hub can be part of a catheter assembly comprising a needle hub having a needle extending distally thereof and through the catheter hub, the reinforcement sleeve and the catheter tube in a ready to use position.

In an example, the reinforcement sleeve can be located around an exterior of the catheter hub. In an alternative example, the reinforcement sleeve can be located within a lumen of the catheter hub.

Another aspect of the present disclosure is a catheter assembly that may include a catheter hub having a hub body defining an interior cavity. The catheter assembly may further include a bushing disposed in the interior cavity. The catheter assembly may further include a catheter tube sleeved over the bushing. The catheter assembly may further include a sleeve sleeved over the bushing. The catheter assembly may further include a needle hub with a needle. The needle may include a needle shaft having a needle lumen and a needle tip. The needle shaft may project through the bushing and the catheter tube in a ready to use position. The sleeve may be disposed over or within the catheter tube and the bushing. The sleeve and the catheter tube may each have a length along the catheter tube that are different.

The sleeve may be disposed over or within the catheter tube. The sleeve may have a larger diameter when the sleeve is over the catheter tube than when the sleeve is within the catheter tube.

The sleeve disposed over the catheter tube can have a distal tip and wherein the distal tip of the sleeve is located proximally of the distal tip of the catheter tube.

The distal tip of the sleeve is tapered. The tapered tip of the sleeve is formed by a tripping process.

The tipping process can be like the tipping process for forming the tapered tip of the catheter tube. In an example, the sleeve can be located over a metal pin, or the metal pin can be located inside the lumen of the sleeve. The sleeve then undergoes a tipping process that includes heating and subjecting the sleeve against a mold, anvil or tool to shape the tip.

In some examples, the catheter tube is first placed over the metal tip, then the sleeve is placed over both the catheter tube and the metal tip. Then the sleeve undergoes the tipping process.

The catheter tube or the sleeve may be in direct contact with the bushing.

The catheter tube or the sleeve may be a heat shrink tube.

The bushing may further include a tapered seat coupled to the catheter tube or the sleeve. The tapered seat may be conical with a wider opening at a proximal end and a relatively narrower opening at a distal end. The bushing may be made from a metal.

In another example, the bushing is plastic. A hydrophobic coating can optionally be applied to portions of the inner and outer surfaces of the bushing to prevent or thwart thrombus formation, phlebitis, or blood clots. In one example, a parylene coating can be applied to at least the inside surface of the elongated distal seat of the bushing. In still other examples, the bushing can be made from a polymer material or a polymer matrix material, such as medical grade silicone rubber.

The catheter assembly may further include a needle guard having a proximal wall with a proximal opening and two resilient arms slidably on the needle shaft.

The catheter assembly may further include a valve for limiting fluid flow through the catheter hub.

A still further aspect of the invention is a catheter assembly comprising a catheter hub having a hub body with an exterior surface and an interior surface defining an interior cavity; a bushing having a distal section and a proximal section disposed in the interior cavity; a catheter tube having a first length, a lumen, and a distal opening sleeved over the bushing; a reinforcement sleeve having a second length sleeved over the bushing, the reinforcement sleeve overlapping with at least a section of the catheter tube at a distal end of the catheter hub; a needle hub with a needle extending out a distal end of the needle hub, the needle comprising a needle shaft having a needle lumen and a needle tip, the needle shaft projecting through the bushing, the catheter tube, and the reinforcement sleeve in a ready to use position.

The first length can be greater than the second length.

The catheter tube or the reinforcement sleeve can be in direct contact with the bushing.

The reinforcement sleeve can be disposed over the catheter tube or within the catheter tube.

The reinforcement sleeve can be made from a heat shrink material, such as made from a heat shrink tube. The heat shrink material can surround part of the exterior of the catheter tube and optionally also at least part of the exterior of the catheter hub.

In an example, 20% to 70% of the second length, or length of the reinforcement sleeve, can extend distally of catheter hub.

In an example, the part of the inner capillary, or outer capillary as the case may be, that extends distally of the distal end of the catheter hub is about 10% to about 85% of the length of the inner capillary, with 25% to 65% being more preferred.

The catheter tube can be heated or cooled and/or the inner capillary can be cooled or heated to facilitate installation.

The reinforcement capillary or sleeve may be made from the same material as the catheter tube. In an example, the inner capillary may be made from a non-metallic material, such as by extrusion, from a polymer material such as, as non-limiting examples, silicone rubber, latex, polyurethane (PUR), polyethylene terephthalate (PET), fluorinated ethylene propylene (FET), or silicone. In some examples, especially when the sleeve is an outer sleeve used externally of the catheter tube, the material can be Teflon. In still other examples, the material may be made from a thermoplastic elastomer, such as thermoplastic urethanes and polyether block amides (PEBA).

In some examples, antimicrobial agents may optionally be incorporated with the reinforcement capillary or sleeve. In an example, antimicrobial metals may be added or combined with the polymer materials and extruded as part of the sleeve. Exemplary antimicrobial metals include precious metals, such as silver, gold, platinum, copper, and zinc. Physiological antimicrobial metal compounds can include oxides and salts of preferably silver and also gold. These agents include silver acetate, silver benzoate, silver carbonate, silver citrate, silver chloride, silver iodide, silver nitrate, silver oxide, silver sulfadiazine, silver sulfate, gold chloride and gold oxide. Platinum compounds such as chloroplatinic acid or its salts (e.g., sodium and calcium chloroplatinate) may also be used. Alternatively, oxides and salts of copper and zinc such as those indicated above for silver may also be used. Preferred physiological antimicrobial metal compounds usable with the preferred piston of the present invention include silver acetate, silver oxide, ionic silver, silver sulfate, gold chloride, and a combination of silver oxide and gold chloride. The amount of antimicrobial agents can be in the range of 2% to 8% by wt/wt ratio of the combined materials. A colorant may also be added with the antimicrobial agents.

A needle guard having a proximal wall with a proximal opening and two resilient arms can be slidably mounted on the needle shaft.

A valve for limiting fluid flow through the catheter hub comprising at least one slit defining at least two flaps can be located inside the catheter hub.

A valve actuator can be located within the catheter hub and proximally of the valve, the valve actuator can comprise a nose section having a fluid passage and at least one plunger element for pushing by a male Luer tip.

A still further aspect of the invention is a method for manufacturing a catheter assembly, The method can comprise forming a catheter hub with a body having an interior surface defining an interior cavity; sleeving a catheter tube having a first length over a bushing; sleeving a reinforcement sleeve having a second length over the bushing; securing the bushing in the interior cavity of the catheter hub; forming a needle hub with a needle comprising a needle shaft having a needle lumen and a needle tip and projecting the needle shaft through the bushing and the catheter tube in a ready to use position.

The method can further comprise locating a needle guard having a proximal wall with a proximal opening and two resilient arms slidably on the needle shaft. The method can comprise removably engaging the needle guard to the interior of the catheter hub.

The method can further comprise placing a valve for limiting fluid flow through the catheter hub within the interior cavity of the catheter hub and placing a valve actuator proximally of the valve.

A still further aspect of the invention is a catheter assembly comprising a catheter hub having a hub body with an exterior surface and an interior surface defining an interior cavity; a bushing having an elongated distal seat and a tapered seat disposed in the interior cavity; a catheter tube having a first length sleeved over the bushing; a reinforcement sleeve having a second length sleeved over the bushing, the second length being shorter than the first length; a needle hub with a needle extending out a distal end, said needle comprising a needle shaft having a needle lumen and a needle tip, the needle shaft projecting through the bushing, the catheter tube, and the reinforcement sleeve in a ready to use position; and wherein the reinforcement sleeve is disposed over the catheter tube or within the catheter tube.

The reinforcement sleeve can be provided with an indicia. The indicia can be a color that differs from the color of the catheter tube or one or more markings.

The reinforcement sleeve can be provided with an antimicrobial agent.

At least part of the catheter hub can be covered by a heat shrink material. For example, the nose section of the catheter hub can be surrounded by a heat shrink material and then activated, such as with heat, to tightly fit around the nose section.

The heat shrink tubing can be made from a range of thermoplastics, including polyolefin, polyvinyl chloride (PVC), Viton, Neoprene, polytetrafluoroethylene (PTFE), polyurethane (PUR), or fluorinated ethylene propylene (FEP), to name a few non-limiting examples.

A needle guard can be provided. The needle guard can have a surface that is located to a side of the needle and wherein the needle tip and the surface are movable relative to one another so that the surface is located distal of the needle tip to block the needle tip from inadvertent needle sticks. The needle can have a change in profile, such as a crimp, to engage a perimeter on the needle guard. Alternatively, the needle guard can cant over to grip the needle in a protective position without the change in profile.

The needle guard can be located in an interior of the catheter hub and can be biased by the needle to engage the needle hub in the ready to use position. In other examples, the needle guard is retained within the catheter hub by a valve opener. For example, the valve opener can have two plunger elements or legs and can have two bands connecting different edges of the two plunger elements to thereby form a continuous section and two through openings. The two elbows of the needle guard can be located in or at the two through openings and be held within the catheter hub.

Where a catheter assembly is provided with a reinforcement capillary or sleeve, the feature can be used as an inner sleeve that fits within the bore of the catheter tube to support the catheter tube from kinking or an outer sleeve that fits around the outside of the catheter tube to support the catheter tube from kinking.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1A is a cross sectional view of an embodiment of an IV catheter assembly provided in accordance with aspects of the present disclosure;

FIG. 1B is a cross sectional view of another embodiment of an IV catheter assembly provided in accordance with aspects of the present disclosure;

FIG. 2 is an isometric view of a catheter hub assembly provided in accordance with aspects of the present disclosure;

FIG. 3 is cross sectional view of the catheter hub assembly of FIG. 2;

FIG. 4 shows an embodiment of a bushing provided in accordance with aspects of the present disclosure in a side view;

FIG. 5 shows the bushing of FIG. 4 attached to a catheter tube;

FIG. 6 shows the bushing of FIG. 4 with the catheter tube in a partially deflected state;

FIG. 7 shows the hub assembly of FIG. 2 with the catheter tube in a partially deflected state;

FIG. 8 shows another embodiment of an IV catheter assembly;

FIG. 9 shows yet another embodiment of an IV catheter assembly; and

FIG. 10 shows still yet another embodiment of an IV catheter assembly.

### DETAILED DESCRIPTION

The detailed description set forth below in connection with the appended drawings is intended as a description of the presently preferred embodiments of needle assemblies and components for use with or forming the needle assemblies provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

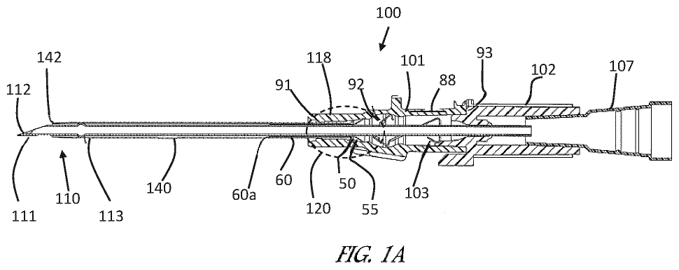

FIG. 1A shows a catheter device or assembly 100 provided in accordance with aspects of the present disclosure. The catheter device 100 can include a catheter hub 101 having a body 88 with an exterior surface and an interior surface defining a hollow interior cavity 92 between a proximal end 93 and a distal end 91, a needle hub 102, and a needle 110 extending from the needle hub 102 and projecting through a flexible tube or catheter tube 140 in a ready to use position. A needle tip 112 with a needle bevel at a distal end of the needle 110 can extend out a distal end opening 142 of the catheter tube 140 in the ready position of FIG. 1A. The needle 110 can be understood to have a wall surface defining a needle shaft 111 having a needle lumen. A push tab can be provided with the hub body 88 to aid in pushing the catheter hub 101 during catheterization and a pair of wings can extend laterally of the hub body 88 to facilitate securing the catheter hub to the patient following successful venipuncture.

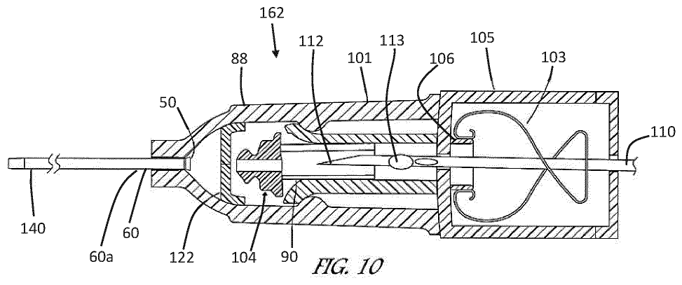

The catheter device 100 can optionally include a needle shield or needle guard 103 to cover the needle tip 112 in a protective position after the needle 110 is withdrawn from the catheter hub 101 following successful venipuncture to prevent needle stick injuries. The needle guard 103 can be secured inside the interior cavity 92 in a ready to use position. The needle guard 103 can alternatively be provided outside or partially outside the interior cavity 92, as further discussed below with reference to FIG. 10. The needle guard 103 can have a proximal wall with a perimeter defining an opening for accommodating the needle, two arms extending distally of the proximal wall, and end surfaces or distal walls for covering the needle tip 112 in a protective position to prevent inadvertent needle sticks.

In one example, the needle guard 103 has a surface that is located to the side of the needle shaft 111 in a ready to use position, and the surface is movable distally of the needle tip 112 in a protective position to cover the needle tip 112 from inadvertent needle sticks. Examples of needle shields can be found in U.S. Pat. No. 8,827,965 and in U.S. patent application Ser. No. 13/257,572, published as US 2012/0046620 A1, the contents of which are expressly incorporated herein by reference. Thus, the needle guard has a surface that is located to a side of the needle and wherein the needle tip and the surface are movable relative to one another so that the surface is located distal of the needle tip to block the needle tip from inadvertent needle sticks. These needle shields or guards can be unitarily formed or can be made separately from multiple parts or components and subsequently assembled together.

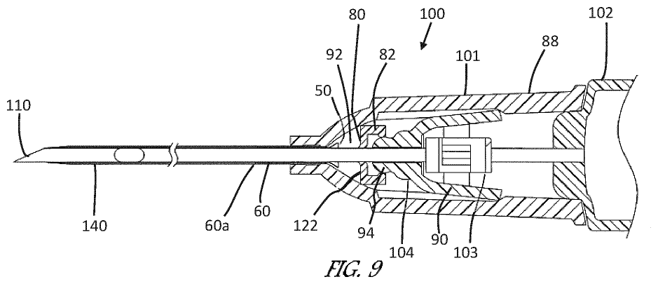

The needle 110 can include a change in profile or contour 113 having a different surface contour or shape than the remaining needle shaft. The change in profile 113 can embody a crimp or a radial bulge incorporated near the needle tip 112 for interacting with the perimeter defining the opening on the proximal wall of the needle guard 103 to stop the needle guard 103 from displacing distally off of the needle 110 in the protective position. Some needle guards 103 can operate without a change in profile 113 on the needle 110, such as ones that can cant or slant so that an opening that surrounds the needle 110 can grip the needle shaft 111 without a change in profile, such as those disclosed in U.S. Pat. No. 6,709,419, the contents of which are expressly incorporated herein by reference. In the embodiment of FIG. 1A, the needle shield 103 is located in the interior cavity 92 of the catheter hub 101 and has the needle 110 passing therethrough, such as through the opening on the proximal wall and between the two arms to bias the two arms outwardly so that the elbows of the two arms engage the annular protrusion in the interior of the catheter hub. In other examples, the needle shield 103 is located outside or substantially outside of the catheter hub 101, such as in a third housing or a guard housing located between the catheter hub and the needle hub. Optionally, the needle guard 103 can be omitted. In other examples, the needle guard is retained within the catheter hub by a valve opener, such as the valve opener shown in FIG. 9. For example, the valve opener can have two plunger elements or legs and can have two bands connecting different edges of the two plunger elements to thereby form a continuous section and two through openings. The two elbows of the needle guard can be located in or at the two through openings and be held within the catheter hub.

Optionally a valve and a valve opener can be included with the catheter hub 101, such as shown in FIG. 9. The needle 110 extending from the needle hub 102 can extend through both the valve opener and the valve in the ready to use position. When included, the valve can be wedged inside the interior cavity to control fluid flow thereacross. The valve opener can be slidably mounted proximally of the valve inside the catheter hub. The valve opener can advance into the valve, which can have one or more slits defining two or more flaps, such as three or four flaps, to open the valve for fluid flow. For example, following successful venipuncture, a male Luer adapter of an IV fluid line can connect to the proximal opening of the catheter hub to advance the valve opener into the valve to deflect the two or more valve flaps to open fluid communication between the IV bag and the catheter tube. Exemplary valves and valve openers are disclosed in U.S. Pat. No. 10,166,370 and in U.S. Pub. No. 2018/0214682, the contents of which are expressly incorporated herein by reference.

The catheter tube 140 can be connected to the distal end of the catheter hub 101 via a bushing 50. The bushing 50 presses a proximal end of the catheter tube 140 between the exterior of the bushing 50 and the interior surface of the catheter hub 101 to retain the catheter tube 140 to the catheter hub 101.

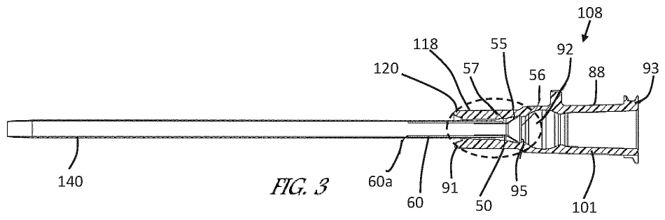

In an example, the bushing 50 can be a bushing that cannot flex, bend, and/or deform. The bushing 50 can be a conventional bushing, such as a conventional metal bushing. The bushing 50 can comprise an anchor 55 or proximal seat 55, which may be called a proximal section with a tapered seat 55 that has a funnel shape, and an elongated distal seat 70 (FIG. 4), which may be called a distal section having an elongated body with a smooth surface, such as a smooth cylindrical body, extending from the tapered seat 55 in a distal direction. In other examples, the shape of the proximal seat 55 is not limited to being tapered or non-tapered. For example, the shape of the proximal seat can have a combination of both tapered and non-tapered features, extended, non-extended, regular, or irregular. The distal seat 70 can be elongated and can embody a hollow cylinder.

In one example and when the bushing 50 is secured to the interior cavity 92 of the catheter hub 101 to wedge the catheter tube 140 to the catheter hub, part of the elongated distal seat 70 can extend at least partly into the bore of the nose section 118 of the catheter hub. In another example, most of the elongated distal seat 70 is recessed within the bore of the nose section 118. The proximal end of the catheter tube 140 is compressed between the exterior of the elongated distal seat 70 and the bore or interior surface of the nose section 118, and possibly also at the proximal seat 55. The area or section of the catheter device 100 in which the bushing 50 wedges the catheter hub against the interior surface of the nose section 118 may be referred to as a securement section 120.

In an example, the securement section 120 can comprise a reinforcement capillary or sleeve 60 in addition to the catheter tube 140. The reinforcement capillary or sleeve 60 can comprise an elongated flexible tube having an exterior and an interior defining a hollow interior. The reinforcement capillary or sleeve 60 can accommodate the catheter tube 140 within the bore of the reinforcement capillary 60 or the reinforcement capillary 60 can be located on the outside or exterior of the catheter tube. In the example of FIG. 1A, the reinforcement capillary 60 is an inner capillary or an inner sleeve 60a, which is understood to be located inside the catheter tube such that the exterior of the reinforcement capillary or sleeve 60 is in contact with the interior of the catheter tube 140.

The inner capillary 60a can be placed inside the bore of the catheter tube 140, and both the catheter tube 140 and the inner capillary 60a can be connected to the distal end of the catheter hub 101 via the bushing 50 at the securement section 120. The bushing 50 can press the proximal end of the catheter tube 140 between the exterior of the inner sleeve 60a and the bore of the nose section 118 of the catheter hub 101 to retain the catheter tube 140 to the catheter hub 101, as well as the inner capillary 60a. Said differently, the bushing 50 can wedge both the catheter tube 140 and the inner capillary 60a against the nose section 118. At least part of the inner sleeve 60a can extend distally of the distal end 91 of the nose section 118. The portion of the inner capillary 60a that extends out the distal end 91 of the catheter hub 101 is flexible and can bend with the catheter tube 140 but its location at the nose section reinforces the catheter tube to deter kinking or collapsing of the catheter tube 140 at or near the nose section of the catheter hub, as further discussed below. In an example, the part of the inner capillary 60a that extends distally of the distal end 91 is about 10% to about 85% of the length of the inner capillary 60a, with 25% to 65% being more preferred. The catheter tube 140 can be heated and/or the inner capillary 60a can be cooled to facilitate installation.

When the catheter tube 140 bends, such as while taping the catheter hub to a patient, the upper side of the bend undergoes tensile stress while the lower side undergoes compressive stress. Kinking occurs when the compressive stress exceeds the catheter tube's yield strength and the catheter tube collapses. The inner capillary 60a can counter forces exerted on the catheter tube 140 due to the bend, such as when tilting the catheter hub against the patient's skin to apply adhesive dressing to secure the catheter hub following successful venipuncture. The inner capillary 60a can increase resistance to bending and can even allow for a relatively larger bending angle prior to any tensile and/or compressive stress failure, or kinking, of the capillary tube 140.

During assembly, the inner capillary 60a can be sleeved over the bushing 50, and the catheter tube 140 can be sleeved over the inner capillary 60a thereafter. Heat may be applied to the catheter tube and/or the inner sleeve to facilitate assembly. Once the inner capillary 60a is placed over the bushing, the inner capillary 60a can be cooled while the catheter tube 140 can be heated to facilitate assembly. The inner capillary 60a may have an inner diameter that is approximately equal to or less than the diameter of the elongated distal seat 70 (FIG. 4) prior to being sleeved over the bushing 50. Once the inner capillary 60a is sleeved over the bushing 50, the inner diameter of the inner capillary 60a may expand to approximately the diameter of the elongated distal seat 70 and apply a compressive force on the elongated distal seat 70, creating a snug fitment. Following the sleeving of both the inner capillary 60a and the catheter tube 140 over the bushing 50, the bushing 50 can then be inserted into the bore of the nose section 118 of the catheter hub 101 to wedge the combination catheter tube and inner capillary to the nose section.

The inner capillary 60a can have a length of approximately 1 cm to 4 cm with 1.5 cm to 2.5 cm being more preferred. The length of the inner capillary 60a can be longer than the length of the elongated distal seat 70, the length of the elongated distal seat 70 being along the direction of the catheter tube 140. The inner capillary 60a may have an inner diameter that can vary depending on the gauge size of the catheter tube. For example, for a G16 metal bushing with an OD of 1.37 mm, the ID of the inner capillary 60a can be 1.34 mm. As another example, a G18 metal bushing with an OD of 1.05 mm, the ID of the inner capillary 60a can be 0.98 mm. In an example, the inner capillary 60a can have a length, and about 15% to about 85% of the length can extend distally of the distal end of the nose section 118. The inner capillary 60a has a shorter length than the length of the catheter tube 140.

The inner capillary 60a may be made from the same material as the catheter tube. In an example, the inner capillary may be made from a non-metallic material, such as by extrusion, from a polymer material such as, as non-limiting examples, silicone rubber, latex, polyurethane (PUR), polyethylene terephthalate (PET), fluorinated ethylene propylene (FET), or silicone. In some examples, especially when the sleeve is an outer sleeve used externally of the catheter tube, the material can be Teflon. In still other examples, the material may be made from a thermoplastic elastomer, such as thermoplastic urethanes and polyether block amides (PEBA).

The needle 110 can extend from a distal end of the needle hub 102 and pass through the interior cavity 92 of the catheter hub 101 through the tapered seat 55 and the elongated distal seat 70 of the bushing 50 and into the catheter tube 140 with the needle tip 112 extending out of the distal end opening 142 of the catheter tube 140 in the ready to use position. The distal portion of the catheter tube 140 can taper inwardly or have an opening that has a size smaller than an outer diameter of the needle 110 to form a seal at the opening of the catheter tube 140 with the needle 110 to prevent fluid from entering the space between the catheter tube 140 and the needle 110 when the needle tip 112 pierces the skin of a patient at an incision or insertion point. The space between the needle 110 and the interior of the catheter tube 140 can be annular and can optionally be sectioned by baffles, such as ridges, or lined with a material that reacts or absorbs with blood to enhance the perception of secondary blood flashback, as further discussed below.

Blood flowing into the needle lumen of the needle 110 and into the needle hub 102 when piercing the skin, such as when entering a vein, is known as primary blood flashback. Blood entering the needle hub 102 can be collected in a vent plug or a blood collection device 107 for sampling. Retraction of the needle tip 112 in a proximal direction into the catheter tube 140 after primary blood flash back can allow fluid or blood to flow into the space between the needle 110 and the interior of the catheter tube 140, known as secondary blood flashback.

An aspect of the invention is therefore understood to include a kink resistant catheter assembly comprising a catheter hub having a hub body with an exterior surface and an interior surface defining an interior cavity. A nose section having a bore at a distal end of the catheter hub. A metal bushing wedging a proximal end of a catheter tube against the interior surface defining the bore of the nose section. A reinforcement capillary or sleeve is disposed internally of the catheter tube. The exterior of the reinforcement capillary or sleeve can contact the interior of the catheter tube or can be spaced from the interior of the catheter tube, at the location that is not wedged by the bushing against the interior of the of the nose section. The reinforcement capillary or sleeve is mounted onto an elongated distal seat of the bushing. The reinforcement capillary or sleeve can terminate before the proximal seat or can contact part of the tapered surface of the proximal seat. The catheter tube is mounted over the reinforcement capillary and the elongated distal seat of the bushing. The metal bushing wedges both the catheter tube and the reinforcement capillary against the interior surface of the bore of the nose section. The inner capillary has a shorter length than the length of the catheter tube. The inner capillary can have a length and about 15% to about 85% of the length can extend distally of the distal end of the nose section of the catheter hub. The inner capillary can counter forces exerted on the catheter tube due to a bend on the catheter tube, such as when tilting the catheter hub against the patient's skin to apply adhesive dressing to secure the catheter hub following successful venipuncture. The inner capillary can increase resistance to bending and can even allow for a relatively larger bending angle prior to any tensile and/or compressive stress failure, or kinking, of the capillary tube.

The kink resistant catheter assembly can further include a needle extending from a needle hub and through the catheter tube and the inner reinforcing sleeve and out a distal opening of the catheter tube. If incorporated, a needle guard can be located inside the interior of the catheter hub or in a third housing located between the catheter hub and the needle hub. The catheter assembly can further optionally include a valve and a valve opener located inside the interior of the catheter hub. The needle guard can locate proximally of the valve. For example, the needle guard can be located between two plunger elements of the valve opener or inside a third housing.

In some examples, antimicrobial agents may optionally be incorporated with the inner reinforcing sleeve 60a, or outer reinforcing sleeve 60b. In an example, antimicrobial metals may be added or combined with the polymer materials and extruded as part of the sleeve. Exemplary antimicrobial metals include precious metals, such as silver, gold, platinum, copper, and zinc. Physiological antimicrobial metal compounds can include oxides and salts of preferably silver and also gold. These agents include silver acetate, silver benzoate, silver carbonate, silver citrate, silver chloride, silver iodide, silver nitrate, silver oxide, silver sulfadiazine, silver sulfate, gold chloride and gold oxide. Platinum compounds such as chloroplatinic acid or its salts (e.g., sodium and calcium chloroplatinate) may also be used. Alternatively, oxides and salts of copper and zinc such as those indicated above for silver may also be used. Preferred physiological antimicrobial metal compounds usable with the preferred piston of the present invention include silver acetate, silver oxide, ionic silver, silver sulfate, gold chloride, and a combination of silver oxide and gold chloride. The amount of antimicrobial agents can be in the range of 2% to 8% by wt/wt ratio of the combined materials. A colorant may also be added with the antimicrobial agents.

FIG. 1B shows a catheter device or assembly 100 provided in accordance with further aspects of the present disclosure. In this embodiment, the reinforcement capillary or sleeve 60 is mounted over the catheter tube 140, or the catheter tube is located inside the bore of the sleeve 60, and the reinforcement sleeve may be referred to as an outer capillary or an outer sleeve 60b. The outer capillary 60b can be placed outside the bore of the catheter tube 140, and both the catheter tube 140 and the outer capillary 60b can be connected to the distal end of the catheter hub 101 via the bushing 50 at the securement section 120. Thus, the outer sleeve 60b can have an interior surface defining a bore and the catheter tube 140 can be located in the bore of the outer sleeve 60b, and possibly contacting the interior surface of the outer sleeve.

The bushing 50 can press the proximal end of the outer capillary 60b between the exterior surface of the catheter tube 140 and the interior of the nose section 118 of the catheter hub 101 at the securement section 120 to partially retain the outer capillary 60b within the catheter hub 101. The retaining forces applied on the outer capillary 60b can also act on the catheter tube 140 positioned between the outer capillary 60b and the bushing 50 to retain the catheter tube 140 in place. The portion of the outer capillary 60b extending out the distal end 91 of the catheter hub 101 can bend with the catheter tube 140 to deter kinking or collapsing of the catheter tube 140 at or near the nose section of the catheter hub. The outer capillary 60b can counter forces exerted on the catheter tube 140 due to the bend, such as when tilting the catheter hub against the patient's skin to apply adhesive dressing to secure the catheter hub following successful venipuncture. The outer capillary 60b can increase resistance to bending and can even allow for a relatively larger bending angle before tensile and/or compressive stress failure, or kinking, of the capillary tube 140.

During assembly, the catheter tube 140 can be sleeved over the bushing 50 and the outer capillary 60b can then be sleeved over both the catheter tube 140 and the bushing 50. Optionally, the outer sleeve 60b can be placed over the catheter tube 140 and the combination then mounted onto the distal seat 70 of the bushing 50. The outer capillary 60b may have an inside or inner diameter that is approximately equal to or less than the outside diameter of the catheter tube 140 prior to being sleeved over the catheter tube 140. In an embodiment, the outer capillary 60b may have an inside diameter that is slightly larger than the outer diameter of the catheter tube. Once the outer capillary 60b is sleeved over the catheter tube 140, the inner diameter of the outer capillary 60b may expand and apply a compressive force on the catheter tube 140 as well as the elongated distal seat 70 of the bushing where the elongated distal seat 70 and the outer capillary 60b overlap. This engagement creates a snug fit or a slight interference fit between the outer capillary 60b and the catheter tube 140. Heat and cooling may be applied to soften the outer sleeve and/or the catheter tube and/or stiffen or shrink the components during assembly. Following mounting of the catheter tube and the sleeve onto the bushing, the bushing 50 can be inserted into the bore of the nose section 118 of the catheter hub 101 to wedge the two against the interior of the nose section.

The outer capillary 60b can have a length of approximately 1 cm to 4 cm with 1.5 cm to 2.5 cm being more preferred. The outer capillary 60b length can be longer than the length of the elongated distal seat 70. The outer capillary 60b can have an inner diameter that can vary depending on the gauge size of the catheter tube.

In an example, the catheter tube 140 can have the same outside diameter along the length thereof except for the two ends, which can have different sizes or shapes. In some examples, the catheter tube 140 can have a first outer diameter and a second outer diameter at the two ends, where the first outer diameter is greater than the second outer diameter. For example, the second outer diameter can be located at the proximal end for fitment with the outer reinforcement sleeve. The second outer diameter can provide room for the outer capillary 60b so that when combined, the overlapped section can have approximately the same OD as other sections of the catheter tube. In other examples, the catheter tube 140 can have a uniform outer diameter and the sleeve is located over the uniform outside diameter. The distal end of the outer sleeve can wedge or taper so that the transition from the catheter tube to the outer sleeve is smooth and capable of penetrating the patient, should the depth of insertion up to the outer sleeve be required for proper venipuncture.

The outer sleeve 60b may be extruded from a non-metallic material, such as a polymer material. The outer sleeve may be made from the same materials as the inner sleeve 60a, discussed above. In some examples, the outer sleeve 60b may be molded or coated with an indicia, such as markings or a different color than the color of the catheter tube. The indicia can act as a “stop” indication to inform the practitioner to not further advance the catheter tube into the patient's skin.

In some examples, the outer reinforcement capillary or sleeve can be made from a heat shrink tubing, which is understood to mean a heat shrinkable non-metallic material having the shape of a tubing. For example, the heat shrink tubing can be placed over the proximal end of the catheter tube, which is located onto the distal seat of a metal bushing, and then the heat shrink tubing is heated, such as with an IR light, a resistance heater, and a heat lamp, to form fit over the catheter tube. The heat shrink tubing can be made from a range of thermoplastics, including polyolefin, polyvinyl chloride (PVC), Viton, Neoprene, polytetrafluoroethylene (PTFE), polyurethane (PUR), or fluorinated ethylene propylene (FEP), to name a few non-limiting examples. The heat shrink tubing and the catheter tube can be wedged within the bore of the nose section of the catheter hub. In alternative embodiments, the heat shrinkable material can, in addition to reinforce the part of the catheter tube adjacent the nose section of the catheter hub, cover the catheter housing. For example, the heat shrinkable material can be placed around the nose section 118 of the catheter tube and over the proximal end of the catheter tube, just distal of the nose section 118 an appropriate length to provide the kinking support. This embodiment, when incorporated, advantageously covers the gap between the catheter hub and the catheter tube, where the catheter tube projects out the hub body 101. Where the heat shrink material also covers the hub body, in addition to part of the catheter tube, the heat shrink material can cover at least part of the nose section of the catheter hub and possibly also part of the hub body proximally of the nose section. This can help to deter blood trapping or blood coagulating at the gap between the catheter tube and the catheter hub.

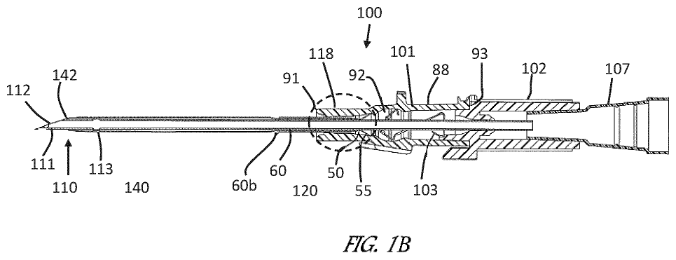

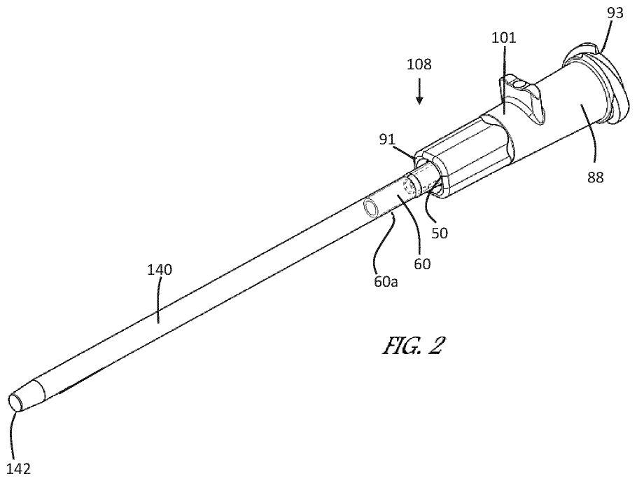

With reference now to FIG. 2, an isometric view of a catheter hub assembly 108 provided in accordance with an aspect of the present disclosure is shown. The catheter hub assembly 108 can include a catheter hub 101, a catheter tube 140, a reinforcement capillary 60, which may be an inner capillary 60a, but can alternatively be an outer capillary 60b, and a bushing 50 located in the interior of the catheter hub. FIG. 3 is a cross-sectional view of the catheter hub assembly 108 of FIG. 2, which more clearly shows the bushing 50 located inside the interior cavity 92 of the catheter hub.

With continued reference to FIG. 3, the catheter hub 101 has an interior cavity 92 defined between an opening at a proximal end 93 of the catheter hub 101 and an opening at a distal end 91 of the catheter hub 101. The bushing 50 can be inserted through the opening at the proximal end 93 of the catheter hub 101 and secured at a distal end of the interior cavity 92 of the catheter hub 101 via press fit, interference fit, adhesive, or combinations thereof. The bushing 50 may also be secured by other fastening means.

The tapered seat 55 and the elongated distal seat 70 can be integrally formed. Alternatively, the tapered seat 55 and the elongated distal seat 70 may be formed as separate pieces that are assembled together by welding or other fastening means. The choice of material of the bushing 50 is of the type that can withstand temperature fluctuations, impacts, corrosive substances, heat, and the atmosphere. In one example, the bushing 50 is metallic. In another example, the bushing 50 is plastic. A hydrophobic coating can optionally be applied to portions of the inner and outer surfaces of the bushing 50 to prevent thrombus formation, phlebitis, or blood clots. In one example, a parylene coating is applied to at least the inside surface of the elongated distal seat 70 to prevent thrombus formation. In still other examples, the bushing can be made from a polymer material or a polymer matrix material, such as medical grade silicone rubber.

The tapered seat 55 can be conical or funnel shaped having a defined thickness with a body having a wider opening at a proximal end 56 and a relatively narrower opening at the distal end 57 of the funnel body. The narrower opening transitions to the elongated distal seat 70. The tapered seat 55 can be rigid and inflexible or semi-rigid to maintain its conical shape after insertion into the catheter hub 101 to thereby wedge the proximal end of the catheter tube 140 to the interior of the catheter hub 101, particularly at the securement section 120. In other examples, the tapered seat 55 can be flexible and elastic.

As discussed above, the tapered seat 55 can be secured inside the interior cavity 92 of the catheter hub by interference fit, press fit or other fastening means. A retaining mechanism or retainer 95 may be provided inside the catheter hub 101 to secure the tapered seat 55 inside the interior cavity 92. For example, the retainer 95 can be a raised circumferential ring or lip formed inside the interior cavity 92 of the catheter hub 101, thus forming a shoulder for the proximal end of the tapered seat 55 to rest against.

The bushing 50 can be inserted into the interior cavity 92 of the catheter hub 101 from the opening at the proximal end 93 of the catheter hub 101 and pushed distally until the tapered seat 55 elastically deforms and slides over the retainer 95 and snaps into a securing position inside the bore and the retainer 95. The raised circumferential ring or shoulder of the retainer 95 can have a bore diameter smaller or slightly smaller than a maximum outside diameter of the wider opening at the proximal end 56 of the tapered seat 55 to provide a positive engagement for the tapered seat 55 against the retainer 95 and prevent the tapered seat 55 of the bushing 50 from sliding proximally out after snapping into position. The circumferential ring can taper gradually inward distally to assist sliding the bushing 50 into the securing position. The circumferential ring can also have a recess or other surface features to resist the distal end 57 of the tapered seat 55 and prevent the bushing 50 from sliding proximally out the interior cavity 92. In other examples, the retainer 95 may be a series of bumps or protrusions or a separately formed element that is inserted into and secured to the catheter hub 101 after installation of the bushing 50.

FIG. 4 shows a side view of a bushing 50 provided in accordance with aspects of the present disclosure. As shown, the elongated distal seat 70 of the bushing 50 can be cylindrical and extend from the narrow opening at the distal end 57 of the tapered seat 55. The elongated distal seat 70 and the tapered seat 55 can be rigid and inflexible or semi-rigid with some flexibility. The distal end or insertion end 68 of the elongated distal seat 70 may be radiused or chamfered to facilitate insertion into the catheter tube or the reinforcement sleeve.

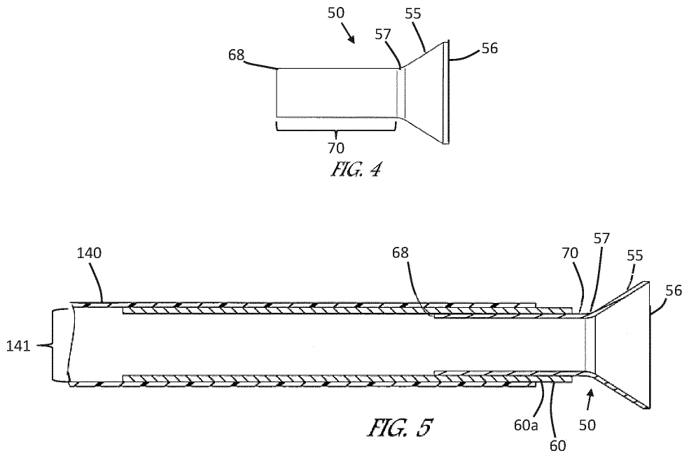

Referring to FIG. 5, the catheter tube 140 and the reinforcement capillary 60, or as shown by example, the inner capillary 60a, can extend to the tapered seat 55, or at least part of the way thereof. In other examples, the catheter tube 140 and the inner capillary 60a can extend to the intersection between the tapered seat 55 and the elongated distal seat 70, or just short of the intersection, such that the proximal end opening of the catheter tube is distal of the proximal end opening of the reinforcement capillary. In other examples, the reinforcement capillary 60 can be an outer sleeve 60b and located externally of the catheter tube 140.

A seal can form at the interface between a lumen 141 of the catheter tube 140, the inner capillary 60a, and the bushing 50 such that fluid cannot flow therebetween the layers or components. A diameter of the elongated distal seat 70 can be the same or larger than the diameter of the lumen 141 of the catheter tube 140 and the inner diameter of the inner capillary 60a. Thus, the catheter tube 140 and the inner capillary 60a can be stretched over the elongated distal seat 70 to form a tight secure fit. Adhesive or other fixing means can be provided to further secure the inner capillary 60a to the elongated distal seat 70 and/or tapered seat 55.

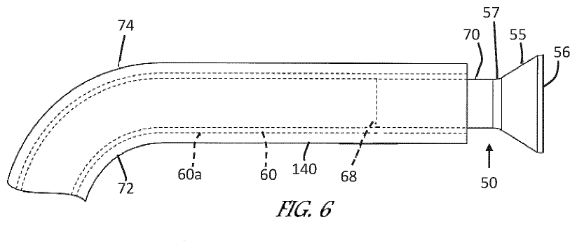

Referring to FIG. 6, the catheter tube 140 and the reinforcement capillary 60, or the inner capillary 60a as shown, can bend together or in unison, forming an n-shape, or whatever words or description that may be used to describe the shape shown. The bending can exert compressive stress at the portion of the catheter tube 140 that forms the inside surface or inside radius 72 of the n-shaped bend and tensile stress at the portion of the catheter tube 140 that forms the outside surface or outside radius 74 of the n-shaped bend. The inner capillary 60a lining or supporting the inside surface 72 and the outside surface 74 of the catheter tube can provide structural support to counter compression of the catheter tube 140 resulting from the bending and reduce possibility of kinking or eliminate kinking. In an example, the n-shape can be characterized as a large radius bend or a tight radius bend. The reinforcement capillary 60 can be an outer capillary 60b and can similarly support the catheter tube 140 from unwanted kinking.

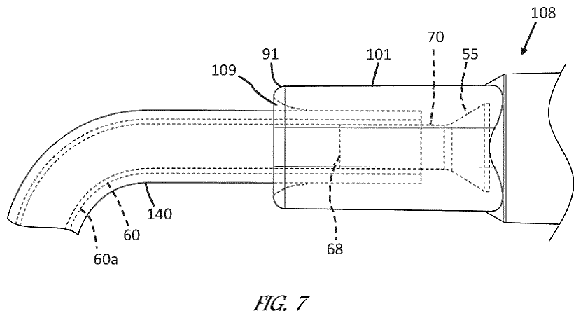

Referring now to FIG. 7, a partial sectional view of the catheter hub assembly 108 of FIG. 2 is shown, with some of the hidden components in dashed-lines for discussion purposes. In the view shown, the distal end 91 of the catheter hub 101 can have a radiused opening forming a recess 109 to accommodate flexing of the catheter tube 140 and the reinforcement capillary 60, or by example, the inner capillary 60a or an outer capillary 60b. The shape of the recess 109 is not limited and can embody any shape provided sufficient clearance is provided to accommodate movement of the catheter tube 140 and the reinforcement capillary 60, such as the distal end 91 shown in FIG. 7.

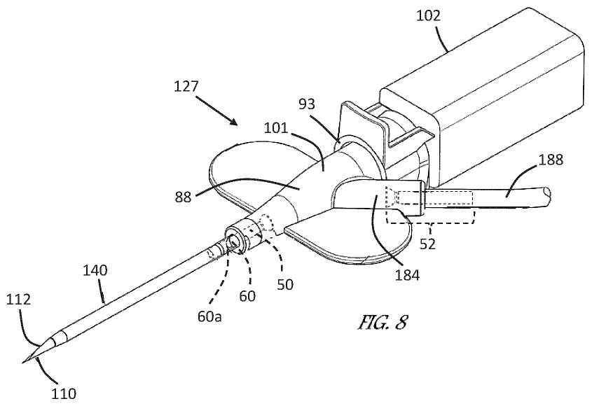

FIG. 8 shows an embodiment of a catheter assembly 127 comprising a catheter hub 101 and a catheter tube 140 attached to bushing 50 described elsewhere herein so that at least part of the reinforcement capillary 60, or the inner capillary 60a as shown but can alternatively be an outer capillary 60b as shown in FIGS. 1B and 5, extends out a distal end of the catheter hub 101. The assembly 127 further comprises a needle hub 102 having a needle 110 extending through the catheter tube 140, a side fluid port 184, and a fluid adaptor (not shown) attached to the side fluid port 184 by a tubing 188, which is attached to the side fluid port 184 by a second bushing 52. The tubing 188 can have a lumen for fluid flow between the side fluid port 184 and the fluid adaptor, which can be a needleless valve having a female Luer inlet. The present catheter assembly may be referred to as an integrated catheter assembly. To prevent the second bushing 52 from being pulled out of the catheter hub 101, the second bushing 52 can be secured to the catheter hub 101 with glue, interference fit, or a combination thereof.

The interior of the catheter hub 101 can be equipped with a septum, a seal or a valve, which can prevent flow out the proximal opening of the hub body after removal of the needle 110 and the needle hub 102. A needle guard 103 as discussed above can be incorporated inside a third housing between the needle hub 102 and the catheter hub 101. The catheter hub 101 is shown with a pair of wings. The needle hub 102 can alternatively have a wing that extends distally along a side of the catheter hub 101 opposite the side fluid port 184, instead of the wing on that side.

A needle tip 112 of the needle 110 can extend distally past a distal opening of the catheter tube 140. Once inserted into the patient, blood flow can be monitored through the needle hub from primary flashback and in the annular space between the needle and the catheter tube 140 from secondary flashback. After successful venipuncture, the needle 110 can be removed from the patient, such as by withdrawing the needle hub 102 in the proximal direction. The seal or septum at the proximal end of the catheter hub can stop fluid flow out the proximal opening. Fluid can be infused through the fluid adaptor (not shown) via the tubing 188 to the side port 184 and then to the catheter hub 101, and then out the catheter tube 140. Alternatively, if the proximal end of the catheter hub 101 has a valve instead of a septum or a seal, then fluid can be infused through the valve via the proximal opening of the hub body and then through catheter tube. A clamp (not shown) can also be used to clamp off the extension line between the side fluid port 184 and the fluid adaptor.

Referring to FIG. 9, the catheter assembly 100 is similar to the catheter assembly of FIG. 1A or FIG. 1B, with either an inner sleeve or an outer sleeve, respectively, except the catheter assembly can further include a valve activator or actuator 104 for opening a valve 122, which can have one or more slits defining a plurality of flaps, such as three, four or more, that can be opened when the valve actuator 104 is advanced by a male medical implement, such as a syringe tip, male Luer connector, or a Luer adaptor. The actuator 104 can have a nose section for physically opening the valve 122 and a plunger end comprising at least one plunger element or leg 90 configured to be pushed by a male medical implement. As shown, the valve actuator 104 has two plunger elements and the needle guard 103 is located therebetween in the ready to use position. Optionally, the valve 122, valve actuator 104, and needle guard 103 can be omitted. The valve 122 is housed within the interior cavity 92 of the catheter hub 101 and when incorporated has the needle 110 projecting therethrough in the ready position, such as shown in FIG. 9.

In some examples, three slits are provided on the valve 122 forming three flaps. In other examples, four slits in the shape of an “X” are provided to form four flaps. Different number of slits and flaps are contemplated. The valve 122 can be seated in a valve seat formed in the interior cavity 92 of the catheter hub. For example, an undercut can be formed in the interior of the catheter hub 101 to retain the valve 122 on the proximal of the vale inside the interior cavity. A shoulder can be provided inside the catheter hub on the distal side of the valve to support the valve from distal displacement. In some examples, bumps or protrusions can be provided around an exterior of the valve 122 to create paths between the valve 122 and the interior of the catheter hub 101 for venting during blood flashback. When the needle 110 is withdrawn from the catheter hub 101 after placement of the flexible tube 14 in the patient's vasculature, the one or more slit closes such that the valve 122 seals upon itself thereby restricting or limiting flow across the valve 122. The valve 122 thus restricts back bleed through the catheter hub 101. The valve 122 can be constructed of a material that forms a seal or a restriction at the interface with the needle 110 and reseals after the needle 110 is withdrawn. For example, and without limitation, the valve 122 can comprise silicone, silicone rubber, polypropylene, or other suitable materials. Unless indicated otherwise, the various components discussed elsewhere herein may be made from conventional materials.

The activator 104 can be provided to press against the valve 122, such as to project through the slits to open or deflect the flaps, when moved distally by a medical implement to open the valve 122 to allow fluid or solution to pass through the valve 122. The activator 104 has a passage formed through the nose section 94 for receiving the needle 110 in the ready position and for fluid flow when the catheter hub is connected to an IV line. The activator can have surface features to provide fluid mixing as fluid enters the catheter hub.

After the needle 110 and needle hub 102 are removed, a male medical implement, such as a Luer tip of a syringe, a male Luer connector or adaptor, such as used in connection with an IV line, a Luer access connector, or a vent plug, can be inserted to push the activator 104 distally into the seal 122 to open the seal 122. For example, the activator 104 can be advanced distally by a syringe tip, which presses against the proximal facing surface of a disc 80 of the valve by the activator 104 to push a nose section 94 of the activator 104 distally forward into the valve 122 inside the skirt 82 to open the one or more slits. In an example, the activator 104 can have a wedge-shaped nose section 94 to press open the valve 122 and an extension, plunger, or leg 90 extending in a proximal direction from the nose section 94 to be pushed against by a male medical implement.

Although a single extension or leg is usable to push the activator 104, two or more leg extensions are preferred, as shown in FIG. 9. The extension 90 can embody one or more separate sections that can be pressed against by a male medical implement to advance the activator 104 against the valve 122. Two spaced apart extensions 90 can be provided to accommodate a needle guard 103 therebetween. Examples of activators can be found in U.S. patent application Ser. No. 14/062,081, 0 published as US 2014/0052065 A1, the contents of which are expressly incorporated herein by reference.

The valve 122 can also be modified or a different valve embodiment may be used with the present catheter assembly. For example, only the disc 80 and not the skirt 82 can be positioned in a seam of a two-piece catheter body. In yet another example, the needle shield 103 can be supported or housed in an intermediate hub between the catheter hub 101 and the needle hub 102, such as shown in FIG. 10. The intermediate hub can be removably coupled with the catheter hub 101 and may be referred to as a third hub or a needle shield or needle guard housing.

In yet other examples, the catheter hub 101 is provided with a valve that can be actuated with fluid pressure only so that the actuator may be omitted from inside the interior cavity of the catheter hub. For example, the valve can flex with head pressure from an IV bag hung on an IV pole pushing up against the valve to open one or more flow paths or channels for fluid flow. In still other examples, the valve is positioned closed to the proximal opening of the catheter hub to be opened by a male Luer connected to the proximal open end of the catheter hub without the actuator or activator 104.

With reference now to FIG. 10, an exemplary catheter assembly 162 is shown, similar to the catheter device of FIG. 9 except that the needle safety shield or guard 103, which comprises a biasing or resilient member, such as a resilient arm, is completely outside or substantially outside of the catheter hub 101. Thus, the present catheter assembly 162 has a catheter tube 140 that is supported by a reinforcement capillary or sleeve, which can be an inner sleeve or an outer sleeve as described elsewhere. As shown, an intermediate hub, a needle shield hub, or a third hub 105 is located, at least in part, between the catheter hub 101 and the needle hub 102. The needle 110 has a change in profile 113, similar to other embodiments discussed elsewhere herein.

The needle shield 103 is located on or in the intermediate hub 105. The intermediate hub 105 can be enclosed as illustrated, can have a single wall, or can have openings in the wall. The needle shield 103 can be supported by support flange 106 of the intermediate hub 105 or can have the distal arms directly touching the needle 110. The support flange 106 extends from a distal wall of the intermediate hub 105 so that the resilient arms of the needle safety shield or guard 103 are supported on the flange 106.

In use, as the needle 110 is retracted as shown and the needle hub is out of view, the change in contour or profile 113 will engage the proximal opening on the proximal wall of the needle shield 103 and pull the needle shield 103 proximally so that the two resilient arms are pulled off the support flange 106 and then close together to block the needle tip 112.

Methods of making and of using the needle devices and their components as discussed elsewhere herein are contemplated.

Although limited embodiments of various catheter assemblies with a bushing and a reinforcement sleeve have been specifically described and illustrated herein, many modifications and variations will be apparent to those skilled in the art. For example, any over the needle catheter can benefit from using the inner or outer capillary disclosed herein to reduce probability of kinking and increase flexibility, allowing the patient to move about without causing further discomfort at or near the insertion point. Furthermore, it is understood and contemplated that features specifically discussed for one catheter assembly with a bushing and a reinforcement sleeve may be adopted for inclusion with another catheter device provided the functions are compatible. Accordingly, it is to be understood that the catheter devices with a bushing and a reinforcement sleeve and their components constructed according to principles of the disclosed devices, systems, and methods may be embodied other than as specifically described herein. The disclosure is also defined in the following claims.

1-22. (canceled)

23. A catheter assembly comprising:
a catheter hub having a hub body with an exterior surface and an interior surface defining an interior cavity;
a bushing having a distal section and a proximal section disposed in the interior cavity;
a catheter tube having a first length, a lumen, and a distal opening sleeved over the bushing;
a reinforcement sleeve, made from a non-metallic material, having a second length sleeved over the bushing, the reinforcement sleeve overlapping with at least a section of the catheter tube at a distal end of the catheter hub to resist or prevent kinking of the catheter tube;
a needle hub with a needle extending out a distal end of the needle hub, the needle comprising a needle shaft having a needle lumen and a needle tip, the needle shaft projecting through the bushing, the catheter tube, and the reinforcement sleeve in a ready to use position.

24. The catheter assembly of claim 23, wherein the first length is greater than the second length.

25. The catheter assembly of claim 23, wherein the catheter tube or the reinforcement sleeve is in direct contact with the bushing.

26. The catheter assembly of claim 23, wherein the reinforcement sleeve is disposed over the catheter tube or within the catheter tube.

27. The catheter assembly of claim 23, wherein the reinforcement sleeve is a heat shrink tube.

28. The catheter assembly of claim 27, wherein the heat shrink tube covers at least part of an exterior of the catheter hub.

29. The catheter assembly of claim 23, wherein 20% to 70% of the second length extends distally of catheter hub.

30. The catheter assembly of claim 23, further comprising a needle guard having a proximal wall with a proximal opening and two resilient arms slidably mounted on the needle shaft.

31. The catheter assembly of claim 23, further comprising a valve for limiting fluid flow through the catheter hub, the valve comprising at least one slit defining at least two flaps.

32. The catheter assembly of claim 31, further comprising a valve actuator located within the catheter hub and proximally of the valve, said valve actuator comprising a nose section having a fluid passage and at least one plunger element for pushing by a male Luer tip.

33. The catheter assembly of claim 23, further comprising a side fluid port extending radially of the hub body, the side fluid port having a bushing securing a tubing to a lumen of the side fluid port.

34. A method for manufacturing a catheter assembly, the method comprising:
forming a catheter hub with a body having an interior surface defining an interior cavity;
sleeving a catheter tube having a first length over a bushing;
sleeving a non-metallic reinforcement sleeve having a second length over the bushing;
securing the bushing in the interior cavity of the catheter hub;
forming a needle hub with a needle comprising a needle shaft having a needle lumen and a needle tip and projecting the needle shaft through the bushing and the catheter tube in a ready to use position.

35. The method of claim 34, further comprising locating a needle guard having a proximal wall with a proximal opening and two resilient arms slidably on the needle shaft.

36. The method of claim 34, further comprising placing a valve for limiting fluid flow through the catheter hub within the interior cavity of the catheter hub and placing a valve actuator proximally of the valve.

37. A catheter assembly comprising:
a catheter hub having a hub body with an exterior surface and an interior surface defining an interior cavity;
a bushing having an elongated distal seat and a tapered seat disposed in the interior cavity;
a catheter tube having a first length sleeved over the bushing;
a non-metallic reinforcement sleeve having a second length sleeved over the bushing, the second length being shorter than the first length;
a needle hub with a needle extending out a distal end of the needle hub, said needle comprising a needle shaft having a needle lumen and a needle tip, the needle shaft projecting through the bushing, the catheter tube, and the reinforcement sleeve in a ready to use position; and
wherein the reinforcement sleeve is disposed over the catheter tube or within the catheter tube.

38. The catheter assembly of claim 37, wherein 20% to 75% of the second length of the reinforcement sleeve extends distally of a nose section of the catheter hub.

39. The catheter assembly of claim 37, wherein the catheter tube or the reinforcement sleeve is in direct contact with the bushing.

40. The catheter assembly of claim 37, wherein the reinforcement sleeve is a heat shrink tube.

41. The catheter assembly of claim 37, wherein the tapered seat is conical and has an opening at a proximal end that is larger than an opening at a distal end.

42. The catheter assembly of claim 37, wherein the reinforcement sleeve is provided with an indicia.

43. The catheter assembly of claim 37, wherein the reinforcement sleeve is provided with an antimicrobial agent.

44. The catheter assembly of claim 37, wherein at least part of the catheter hub is covered by a heat shrink material.
